SEQUENCE LISTING (7 sequences)

SEQ ID NO: 1 - RNA, 25 nt, synthetic construct
  source 1..25: mol_type = other RNA
ataatggctggattgtttaaaaaaa

SEQ ID NO: 2 - RNA, 27 nt, synthetic construct
  source 1..27: mol_type = other RNA
tttttttaaacaatccagccattattc

SEQ ID NO: 3 - RNA, 25 nt, synthetic construct
  source 1..25: mol_type = other RNA
catcaagacatcgtgcgatatttgc

SEQ ID NO: 4 - RNA, 27 nt, synthetic construct
  source 1..27: mol_type = other RNA
gcaaatatcgcacgatgtcttgatgtc

SEQ ID NO: 5 - DNA, 16 bp, synthetic construct
  source 1..16: mol_type = other DNA
gatttagctctgctct

SEQ ID NO: 6 - DNA, 16 bp, synthetic construct
  source 1..16: mol_type = other DNA
ctcatgccattccttt

SEQ ID NO: 7 - DNA, 16 bp, synthetic construct
  source 1..16: mol_type = other DNA
gtgaagctaagaagaa

(19) United States
(12) Patent Application Publication
(10) Pub. No.: US 2026/0117231 A1
(43) Pub. Date: Apr. 30, 2026

(54) COMPOSITIONS AND METHODS FOR TREATING MYOPATHY

(71) Applicant: THE TRUSTEES OF COLUMBIA UNIVERSITY IN THE CITY OF NEW YORK, New York, NY (US)

(72) Inventors: Umrao R. Monani, Oradell, NJ (US); Narendra N. Jha, New York, NY (US)

(21) Appl. No.: 19/422,180

(22) Filed: Dec. 16, 2025

Related U.S. Application Data
(63) Continuation of application No. PCT/US2024/035097, filed on Jun. 21, 2024.
(60) Provisional application No. 63/510,059, filed on Jun. 23, 2023.

(51) Int. Cl. C12N 15/113; A61P 21/00

(52) U.S. Cl. CPC C12N 15/113; A61P 21/00; C12N 2310/11; C12N 2310/14; C12N 2310/341; C12N 2310/531

(57) ABSTRACT

The present disclosure provides, inter alia, compositions and methods for treating premature aging of muscle stem cells to combat myopathy by targeting Cdkn2a.

### CROSS REFERENCE TO RELATED APPLICATIONS

This application is a continuation of PCT International Application No. PCT/US2024/035097, filed on Jun. 21, 2024, which claims priority to U.S. Provisional Application No. 63/510,059, filed on Jun. 23, 2023. The entire contents of the aforemetioned applications are incorporated by reference herein.

### FIELD OF DISCLOSURE

The present disclosure provides, inter alia, compositions and methods for treating skeletal muscle diseases in a subject by treating with agents that inhibit expression or function of Cdkn2a.

### INCORPORATION BY REFERENCE OF SEQUENCE LISTING

This application contains references to amino acids and/or nucleic acid sequences that have been filed concurrently herewith as sequence listing XML file “CU23330-seq.xml”, file size of 7,235 bytes, created on Jul. 19, 2024. The aforementioned sequence listing is hereby incorporated by reference in its entirety pursuant to 37 C.F.R. § 1.52 (e) (5).

### BACKGROUND

Spinal muscular atrophy (SMA) is a frequently fatal pediatric neuromuscular disorder caused by low levels of the SMN protein. SMA has traditionally been defined as a spinal motor neuron disease. Indeed, currently available SMN repletion treatments for the disease focus mostly on restoring the protein to spinal motor neurons. Yet, it is now clear that muscle dysfunction in the disease is not merely a consequence of motor neuron degeneration. Rather, SMN is intrinsically important in muscle. In other words, low SMN in muscle triggers muscle dysfunction in a cell-autonomous manner. It has been determined that these defects derive, at least in part, from defective muscle stem cells (satellite cells).

Currently available SMN repletion treatments for SMA have either deliberately or unwittingly ignored the importance of restoring the protein to muscle. Spinraza®, a splice-switching antisense oligonucleotide (ASO) and the first of the SMN-enhancing agents to enter the clinic is administered intrathecally and therefore restricted to the CNS5, while Zolgensma® (AAV9-SMN), which is promoted as, and will likely remain a one-time treatment (owing to development of neutralizing antibodies in treated patients), fails to consider the phenomenon of myofiber turnover and concomitant loss of the SMN plasmid that would eventually turn transduced muscle back to an SMA-like state. Risdiplam®, a small-molecule splice modulator of SMN2 and the most recent addition to the arsenal of SMA treatments is approved for chronic use. However, it augments SMN levels only modestly (˜2.5-fold), with estimates suggesting that despite treatment, type I SMA patients only express ˜30% of WT SMN levels-well below the heterozygous concentrations required to ensure a disease-free state. Risdiplam's modest effect raises questions about its ability to permanently stave off disease.

It is unclear if current SMA treatments target satellite cells, suggesting that there is potential for improved treatments that would specifically mitigate muscle damage in the disease. It has also been determined that defects in SMA satellite cells likely arise from untimely senescence-premature aging. Thus, there exists a need to prevent the premature aging of muscle satellite cells as a means of combating muscle disease in SMA and other muscular dystrophies such as Duchenne Muscular Dystrophy (DMD). The compositions and methods disclosed herein address these and other needs.

### SUMMARY

According to some aspects, the present disclosure provides a method of treating or ameliorating myopathy in a subject in need thereof comprising the step of administering to the subject an effective amount of an inhibitor of Cdkn2a. In some embodiments, the myopathy is a neuromuscular disease. In some embodiments, the myopathy is sarcopenia. In some embodiments, the myopathy is spinal muscular atrophy (SMA). In some embodiments, the myopathy is Duchenne Muscular Dystrophy (DMD). In some embodiments, the inhibitor of Cdkn2a is one or more oligonucleotide therapeutic agents. In some embodiments, the inhibitor of Cdkn2a is one or more of RNAi and antisense oligonucleotides. In some embodiments, the inhibitor of Cdkn2a is one or more of siRNAs, shRNAs and Gapmers. In some embodiments, the inhibitor of Cdkn2a is a small molecule antagonists/inhibitor. In some embodiments, the inhibitor of Cdkn2a is effective to restore the ability of self-renewal in one or more muscle satellite cells. In some embodiments, the inhibitor of Cdkn2a is effective to prevent premature aging in one or more muscle satellite cells. In some embodiments, the method of treating or ameliorating myopathy in a subject further comprises the step of administering one or more SMN repletion therapies.

According to some aspects, the present disclosure provides a method of preventing premature aging of muscle satellite cells in a subject comprising the step of administering to the subject an effective amount of an inhibitor of Cdkn2a. In some embodiments, the inhibitor of Cdkn2a is one or more oligonucleotide therapeutic agents. In some embodiments, the inhibitor of Cdkn2a is one or more of RNAi and antisense oligonucleotides. In some embodiments, the inhibitor of Cdkn2a is one or more of siRNAs, shRNAs and Gapmers. In some embodiments, the inhibitor of Cdkn2a is a small molecule antagonists/inhibitor. In some embodiments, the inhibitor of Cdkn2a is effective to treat or ameliorate a myopathy. In some embodiments, the inhibitor of Cdkn2a is effective to treat or ameliorate one or more of a neuromuscular disease or of sarcopenia. In some embodiments, the inhibitor of Cdkn2a is effective to treat or ameliorate one or more of spinal muscular atrophy (SMA) and Duchenne Muscular Dystrophy (DMD).

According to some aspects, the present disclosure provides, a method of restoring self-renewal potential of muscle satellite cells in a subject comprising the step of administering to the subject an effective amount of an inhibitor of Cdkn2a. In some embodiments, the inhibitor of Cdkn2a is one or more oligonucleotide therapeutic agents. In some embodiments, the inhibitor of Cdkn2a is one or more of RNAi and antisense oligonucleotides. In some embodiments, the inhibitor of Cdkn2a is one or more of siRNAs, shRNAs and Gapmers. In some embodiments, the inhibitor of Cdkn2a is a small molecule antagonists/inhibitor. In some embodiments, the restoring of self-renewal by administration of inhibitor of Cdkn2a is effective to treat or ameliorate a myopathy. In some embodiments, the restoring of self-renewal by administration of inhibitor of Cdkn2a is effective to treat or ameliorate one or more of a neuromuscular disease or of sarcopenia. In some embodiments, the restoring of self-renewal by administration of inhibitor of Cdkn2a is effective to treat or ameliorate one or more of spinal muscular atrophy (SMA) and Duchenne Muscular Dystrophy (DMD).

According to some aspects, the present disclosure provides a composition for use in preventing premature aging or restoring self-renewal of muscle satellite cells in a subject, the composition comprising an inhibitor of Cdkn2a. In some embodiments, the inhibitor of Cdkn2a is one or more oligonucleotide therapeutic agents. In some embodiments, the inhibitor of Cdkn2a is one or more of RNAi and antisense oligonucleotides. In some embodiments, the inhibitor of Cdkn2a is one or more of siRNAs, shRNAs and Gapmers. In some embodiments, the inhibitor of Cdkn2a is a small molecule antagonist/inhibitor. In some embodiments, the composition further comprises one or more SMN repletion therapies.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D show and describe disease phenotype in mutants selectively depleted of SMN in muscle and harboring 1 copy of the SMN2 gene. (FIG. 1A) Reduced lifespan of mutants. P<0.001, log-rank test. (FIG. 1B) Mutants perform poorly in a righting reflex test. *, *** P<0.05 and P<0.001, t test. (FIG. 1C) PND10 mutant exhibiting hindlimb paralysis (arrows). (FIG. 1D) Intercostal muscle in mutants contains fewer myofibers many of which harbor central nuclei (arrows).

FIGS. 2A-2F show and describe Overt disease is observed in 6-month-old MyoD-iCre SMA mutants with 2 SMN2 copies. (FIG. 2A) Mutants perform poorly on the rotarod and in a grip strength assay. (FIG. 2B) Mutant muscle does not contract as forcefully as Ctrl muscle does. (FIG. 2C) H&E-stained transverse sections of gastrocnemius muscle show extensive myofiber pathology in the mutant. Hypotrophic fibers (solid arrows), regenerating fibers with cytoplasmic basophilia (open arrowhead), split fibers (solid arrowheads) or degenerating fibers (open arrows) are shown. (FIG. 2D) AChR clusters in mutants are fragmented. (FIG. 2E) Extent of NMJ fragmentation in EDL muscle from mutants and controls. (FIG. 2F) Mutant lifespan is considerably shortened. Note: *, ***, P<0.05, P<0.01, t test or one-way ANOVA.

FIGS. 3A-3B show and describe evidence of a contributing effect of satellite cells to muscle pathology in SMA. (FIG. 3A) Restricting SMN depletion to mature myofibers using the HSA-Cre driver reduces disease severity in mutant mice and prolongs survival as depicted in the Kaplan-Meier survival curve. Mutants in which depletion is effected in satellite cells and myofibers using the MyoD-iCre driver are severely affected. (FIG. 3B) Examination of muscle in 6-month-old MyoD-iCre; SMN2tg/tg; SmnF7/− mutants indicates far greater numbers of myofibers with centralized nuclei relative to degenerating fibers (as assessed by leakage of serum IgG into cells). *, **, P<0.05 and P<0.01, t test, n=4.

FIGS. 4A-4B show and describe a senescence gene signature in SMA muscle. (FIG. 4A) Cdkn2a (p16^{INK4a}) is increased in adult SMA but not ALS muscle. (FIG. 4B) Altered expression of other genes associated with and known to be involved with regulation of the senescent phenotype in stem cells. Note: *, **, ***, P<0.05, 0.01 and 0.001 respectively, t test, n≥4.

FIG. 5 shows and describes abnormal Cdkn2a activity in SCs. Immunohistochemistry on transverse sections of mutant muscle depicts abnormal Cdkn2a activity in satellite cells. Arrows highlight representative dual-stained foci.

FIGS. 6A-6C show and describe efficient knockdown of Cdkn2a locus activity. (FIG. 6A) Cdkn2a RNA levels are significantly reduced in cultured cells treated with Gapmer or siRNA. (FIG. 6B) Protein levels are similarly reduced. (FIG. 6C) Western blot corresponding to graph in panel B. Note: ***, P<0.001, one-way ANOVA, n≥4 experiments.

FIG. 7 shows and describes efficient knockdown of Cdkn2a locus activity in a mouse model of muscular dystrophy. Note: ****, P<0.0001, t test, n≥4 experiments.

FIGS. 8A-8D show and describe phenotypes of MyoD-Cre SMA mutant mice with two copies of SMN2 at 7 months. (FIG. 8A) Transverse section of H&E-stained gastrocnemius muscles at 7 months of control and MyoD-Cre SMA mutant with two copies of SMN2. The arrows indicate centralized nuclei. Scale bars: 50 μm. (FIG. 8B) Mean fiber cross-sectional-area, (FIG. 8C) fibers with centralized nuclei and (FIG. 8D) quantification of IgG-positive damaged fibers from the gastrocnemius, triceps and intercostal muscle in control and mutant at 7 months. * p<0.05, unpaired t-test.

FIGS. 9A-9C show and describe quantification of satellite cells in MyoD-Cre SMA mutant with two copies of SMN2 at 6 months. (FIG. 9A) Pax7-stained (Red) transverse section of gastrocnemius muscles at 6 months from control and MyoD-Cre SMA mutant mice with two copies of SMN2. Nuclei were stained with DAPI (4′,6-diamidino-2-phenylindole). Quantification of pax7+ve satellite cells/100 myofiber sections in (FIG. 9B) gastrocnemius and (FIG. 9C) triceps muscles of controls and mutant at 6 months. * p<0.05, paired t-test.

FIG. 10 shows and describes transcript level of cell cycle regulator genes in the gastrocnemius muscles from control and MyoD-Cre SMA mutant mice at symptomatic (6-month) and pre-symptomatic (PND-21) ages.

FIGS. 11A-11C show and describe quantification of satellite cells in MyoD-Cre SMA mutant with two copies of SMN2 at PND21 (presymptomatic age). (FIG. 11A) Pax7-stained (Red) transverse section of gastrocnemius muscles from control and MyoD-Cre SMA mutant mice with two copies of SMN2. Nuclei were stained with DAPI. Quantification of Pax7+ve satellite cells/100 myofiber sections in (FIG. 11B) gastrocnemius and triceps muscles of controls and mutant at PND21. (FIG. 11C) Transcript level of selected cell cycle genes in the gastrocnemius muscles from control and MyoD-Cre SMA mutant mice with two copies of SMN2 at PND21.

FIGS. 12A-12B show and describe quantification of (FIG. 12A) satellite cell number and (FIG. 12B) transcript level of cell cycle regulator (Cdkn2) in MyoD-Cre, and HSA-Cre SMA mutants with two copies of SMN2 and in the DMD-Mdx mice at 6 months.

FIGS. 13A-13C show and describe knockdown of Cdkn2 by two vivo-gapmers in MELDS19 (75 μM) determined by (FIG. 13A) western blot and (FIG. 13B) q-PCR. (FIG. 13C) Transcript level of Cdkn2 in the gastrocnemius muscle of DMD-Mdx mice injected with 20 mg/kg of gapmers.

FIGS. 14A-14C show and describe (FIG. 14A) mutant and control littermate (FIG. 14B) body weight, and (FIG. 14C) survival curve of MyoD-cre (Smn depleted in muscle-stem cells) and HSA-cre (Smn depleted in myofibers) mice.

FIG. 15 shows a depiction of Cdkn2a Gapmer treatment regimen in SMA model mice and relevant controls. Following three rounds of injections, muscle tissue was extracted for analysis.

FIGS. 16A-16C show data of an antisense gapmer against Cdkn2a efficiently restores expression of the gene locus and its downstream targets. (FIG. 16A) Relative levels of Cdkn2a in muscle tissue of healthy controls, sham-treated spinal muscular atrophy (SMA) model mice and gapmer-treated SMA mice. Mice were treated with 20 mg/kg of gapmer at PND5, PND10 and PND17. Tissue was extracted for analysis at PND25. Relative levels of (FIG. 16B) Ccng1, and (FIG. 16C) Perp, downstream targets of Cdkn2a in the same cohort of mice. In each case expression tended to be restored to control levels. Note: *, **, P<0.05 and P<0.01 respectively, n ≥3 mice of each cohort, one-way ANOVA or t tests.

FIGS. 17A-17G show and describe knockdown of Cdkn2a (p16^{INK4a}) in SMA model mice. (FIG. 17A) Single myofiber analysis of controls and mutants depleted of SMN in muscle shows significant numbers of abnormal central nuclei in mutant fibers (asterisk). Nuclei were stained with DAPI or DAPI and H & E (Hematoxylin and Eosin). Single myofibers from mutants have (FIG. 17B) significantly smaller diameters, (FIG. 17C) reduced myonuclear domains and (FIG. 17D) greater numbers of myonuclei per unit length. (FIG. 17E) Reduced pathology in single myofibers from mutants treated with an anti-p16^{INK4a} Gapmer; note fewer central nuclei in Gapmer-treated vs. PBS-treated fibers (asterisks). Correspondingly, (FIG. 17F) myonuclear domain and (FIG. 17G) myofiber diameters are significantly enhanced in mice treated with Gapmer. Note: ***, P <0.001, t test, n=100 fibers from n ≥3 mice of each genotype.

### DETAILED DESCRIPTION OF THE DISCLOSURE

The present disclosure provides, among other things, methods and compositions for the treatment or amelioration of myopathies. In particular, the disclosure relates to the use of an inhibitor of Cdkn2a to prevent premature aging of muscle satellite cells and restore their self-renewal potential. This approach may be beneficial in treating a variety of myopathies, including but not limited to neuromuscular diseases, sarcopenia, spinal muscular atrophy (SMA), and Duchenne Muscular Dystrophy (DMD). The inhibitor of Cdkn2a may be an oligonucleotide therapeutic agent, such as RNAi or antisense oligonucleotides, or a small molecule antagonist or inhibitor. In some cases, the methods and compositions disclosed herein may be used in conjunction with one or more SMN repletion therapies. The disclosure further provides compositions comprising an inhibitor of Cdkn2a for use in preventing premature aging or restoring self-renewal of muscle satellite cells in a subject.

In some aspects, the myopathy treated or ameliorated by the methods disclosed herein may be a neuromuscular disease. Neuromuscular diseases are a broad group of disorders that affect the peripheral nervous system, which includes the muscles and the nerves that control them. These diseases can cause problems with muscle function, such as weakness, cramping, stiffness, and balance issues, and may also affect nerve function, leading to symptoms like numbness, tingling, and pain.

In some cases, the neuromuscular disease may be spinal muscular atrophy (SMA) or Duchenne Muscular Dystrophy (DMD). SMA is a genetic disease affecting the part of the nervous system that controls voluntary muscle movement. It is caused by a loss of specialized nerve cells, called motor neurons, in the spinal cord and the part of the brain connected to the spinal cord (the brainstem). This leads to weakness and wasting (atrophy) of muscles used for activities such as crawling, walking, sitting up, and controlling head movement.

On the other hand, DMD is a genetic disorder characterized by progressive muscle degeneration and weakness due to the alterations of a protein called dystrophin that helps keep muscle cells intact. DMD primarily affects boys and symptoms usually begin in early childhood, typically between ages 2 and 3. Both SMA and DMD can lead to severe physical disability and, in many cases, early death.

The methods and compositions disclosed herein may be particularly beneficial for treating or ameliorating these types of neuromuscular diseases by inhibiting the expression or function of Cdkn2a, thereby preventing premature aging of muscle satellite cells and restoring their self-renewal potential. This may result in improved muscle function and a slowing or halting of disease progression.

In some aspects, the present disclosure provides a method of treating or ameliorating myopathy in a subject. This method may involve administering to the subject an effective amount of an inhibitor of Cdkn2a. The term “myopathy” as used herein refers to any disease or abnormal condition affecting the muscle tissue. The myopathy may be a primary myopathy, which is a disease intrinsic to the muscle tissue, or a secondary myopathy, which is a disease due to a systemic condition that affects the muscle tissue.

The inhibitor of Cdkn2a may be administered in a variety of ways, including but not limited to, oral administration, intravenous injection, intramuscular injection, subcutaneous injection, intraperitoneal injection, intrathecal injection, or direct delivery to the target tissue. The effective amount of the inhibitor of Cdkn2a may vary depending on various factors, including but not limited to, the specific type of myopathy being treated, the severity of the myopathy, the age, weight, and overall health of the subject, and the specific type of inhibitor of Cdkn2a being administered.

In some embodiments, the inhibitor of Cdkn2a may be an oligonucleotide therapeutic agent. Oligonucleotide therapeutic agents are short sequences of nucleotides that can bind to specific sequences of RNA or DNA, thereby inhibiting the expression or function of a specific gene. In some embodiments, the oligonucleotide therapeutic agent may be an RNAi molecule, an antisense oligonucleotide, a siRNA, a shRNA, or a Gapmer. Each of these types of oligonucleotide therapeutic agents may inhibit the expression or function of Cdkn2a in a different manner, and the specific type of oligonucleotide therapeutic agent used may depend on various factors, including but not limited to, the specific type of myopathy being treated and the method of administering the oligonucleotide therapeutic agent. In some embodiments, the siRNA and/or Gapmer is one or more of the sequences of Table 1 below:

TABLE 1

| Notation | Sequence |
|---|---|
| CdKn2a- | Duplex Sequences |
| SiRNA- | 5′-rArUrArArUrGrGrCrUrGrGrArUrUrGrUrUr |
| 13.1 | UrArArArArAAA-3′ (SEQ ID NO: 1) |
| | 5′-rUrUrUrUrUrUrUrArArArCrArArUrCrCrAr |
| | GrCrCrArUrUrArUrUrC-3′ (SEQ ID NO: 2) |
| CdKn2a- | Duplex Sequences |
| SiRNA- | 5′-rCrArUrCrArArGrArCrArUrCrGrUrGrCrGr |
| 13.3 | ArUrArUrUrUGC-3′ (SEQ ID NO: 3) |
| | 5′-rGrCrArArArUrArUrCrGrCrArCrGrArUrGr |
| | UrCrUrUrGrArUrGrUrC-3′ (SEQ ID NO: 4) |
| CdKn2a- | Product sequence 5′-3′ |
| Vivo- | G*A*T*T*T*A*G*C*T*C*T*G*C*T*C*T |
| Gapmer-1 | (SEQ ID NO: 5) |
| CdKn2a- | Product sequence 5′-3′ |
| Vivo- | C*T*C*A*T*G*C*C*A*T*T*C*C*T*T*T |
| Gapmer-2 | (SEQ ID NO: 6) |
| CdKn2a- | Product sequence 5′-3′ |
| Vivo- | G*T*G*A*A*G*C*T*A*A*G*A*A*G*A*A |
| Gapmer-3 | (SEQ ID NO: 7) |

In some embodiments, the inhibitor of Cdkn2a may be a small molecule antagonist or inhibitor. Small molecule antagonists or inhibitors are typically low molecular weight organic compounds that can bind to a specific target protein and inhibit its function. The specific type and amount of small molecule antagonist or inhibitor used may depend on various factors, including but not limited to, the specific type of myopathy being treated, the severity of the myopathy, and the age, weight, and overall health of the subject.

In some aspects, the method of treating or ameliorating myopathy in a subject may further comprise administering one or more SMN repletion therapies. SMN repletion therapies are treatments that increase the levels of the survival motor neuron (SMN) protein, which is deficient in subjects with spinal muscular atrophy (SMA). The specific type and amount of SMN repletion therapy used may depend on various factors, including but not limited to, the specific type of myopathy being treated, the severity of the myopathy, and the age, weight, and overall health of the subject.

In some aspects, the myopathy treated or ameliorated by the methods disclosed herein may be sarcopenia. Sarcopenia is a disease associated with the aging process, characterized by loss of muscle mass and function. This condition can lead to physical disability, poor quality of life, and death. In some embodiments, the inhibitor of Cdkn2a is beneficial for treating or ameliorating sarcopenia by preventing premature aging of muscle satellite cells and restoring their self-renewal potential. This results in improved muscle function and a slowing or halting of disease progression.

In some embodiments, the inhibitor of Cdkn2a may be an oligonucleotide therapeutic agent. Oligonucleotide therapeutic agents are short sequences of nucleotides that can bind to specific sequences of RNA or DNA, thereby inhibiting the expression or function of a specific gene. In some cases, the oligonucleotide therapeutic agent may be an RNAi molecule. RNAi, or RNA interference, is a biological process in which RNA molecules inhibit gene expression or translation by neutralizing targeted mRNA molecules.

In other embodiments, the oligonucleotide therapeutic agent may be an antisense oligonucleotide. Antisense oligonucleotides are short, single-stranded DNA or RNA molecules that can bind to specific mRNA molecules and prevent them from being translated into protein.

In yet other embodiments, the oligonucleotide therapeutic agent may be a siRNA, a shRNA, or a Gapmer. siRNAs, or small interfering RNAs, are double-stranded RNA molecules that can induce the degradation of specific mRNA molecules, thereby inhibiting their translation into protein. shRNAs, or short hairpin RNAs, are similar to siRNAs but are formed from a single strand of RNA that folds back on itself to form a hairpin structure. Gapmers are antisense oligonucleotides that contain a central block of deoxynucleotides flanked by blocks of modified nucleotides, and are designed to induce RNase H-mediated degradation of the target RNA. In some embodiments, the siRNA and/or Gapmer is one or more of the sequences of Table 1. In some embodiments, the oligonucleotide therapeutic agent comprises one or more of SEQ ID NOs: 1-7.

The specific type and amount of oligonucleotide therapeutic agent used may depend on various factors, including but not limited to, the specific type of myopathy being treated, the severity of the myopathy, the age, weight, and overall health of the subject.

In some embodiments, the oligonucleotide therapeutic agent may be administered in combination with one or more other therapeutic agents, such as SMN repletion therapies or other treatments for myopathy. The combination of a oligonucleotide therapeutic agent with other therapeutic agents may provide a synergistic effect, resulting in a more effective treatment or amelioration of myopathy.

In some aspects, the inhibitor of Cdkn2a may be characterized as a small molecule antagonist or inhibitor. Small molecule antagonists or inhibitors are typically low molecular weight organic compounds that can bind to a specific target protein and inhibit its function. These small molecules can interfere with the biological function of Cdkn2a by binding to its active site or to allosteric sites, thereby preventing the normal operation of the protein.

In some cases, the small molecule antagonist or inhibitor of Cdkn2a may be selected from a group of compounds known to inhibit the function of Cdkn2a. These compounds may include, but are not limited to, 4-(Methylnitrosamino)-1-(3-pyridyl)-1-butanone, 5-Aza-2′deoxycytidine, 5-fluorouracil, 5-Methylcytosine, 5-metylthioadenosine, 7,12-dimethylbenz (a) anthracene, acnu, Aflatoxin B1, Agar, and Agarose. The specific type and amount of small molecule antagonist or inhibitor used may depend on various factors, including but not limited to, the specific type of myopathy being treated, the severity of the myopathy, and the age, weight, and overall health of the subject.

In some embodiments, the small molecule antagonist or inhibitor of Cdkn2a may be administered in combination with one or more other therapeutic agents, such as SMN repletion therapies or other treatments for myopathy. The combination of a small molecule antagonist or inhibitor of Cdkn2a with other therapeutic agents may provide a synergistic effect, resulting in a more effective treatment or amelioration of myopathy.

In some cases, the small molecule antagonist or inhibitor of Cdkn2a may be administered in a variety of ways, including but not limited to, oral administration, intravenous injection, intramuscular injection, subcutaneous injection, intraperitoneal injection, intrathecal injection, or direct delivery to the target tissue. The effective amount of the small molecule antagonist or inhibitor of Cdkn2a may vary depending on various factors, including but not limited to, the specific type of myopathy being treated, the severity of the myopathy, the age, weight, and overall health of the subject, and the specific type of inhibitor of Cdkn2a being administered.

In some aspects, the inhibitor of Cdkn2a may be effective to restore the ability of self-renewal in one or more muscle satellite cells. Muscle satellite cells are a type of stem cell found in skeletal muscle tissue. These cells play a central role in muscle repair and regeneration, as they have the ability to self-renew and differentiate into mature muscle cells. However, in conditions such as myopathy, the ability of these cells to self-renew and contribute to muscle regeneration may be compromised.

In some cases, the compromised self-renewal ability of muscle satellite cells may be due to premature aging, which can be triggered by increased expression or activity of Cdkn2a. In the context of muscle satellite cells, increased Cdkn2a activity can lead to premature aging of these cells, thereby impairing their ability to self-renew and contribute to muscle regeneration.

By inhibiting the expression or function of Cdkn2a, the methods and compositions disclosed herein may be able to restore the self-renewal ability of muscle satellite cells. This could be achieved, for example, by using an oligonucleotide therapeutic agent or a small molecule antagonist or inhibitor that targets Cdkn2a. In some embodiments, the oligonucleotide therapeutic agent may be an RNAi molecule, an antisense oligonucleotide, a siRNA, a shRNA, or a Gapmer, each of which can bind to the Cdkn2a mRNA and prevent its translation into protein. In some embodiments, the siRNA and/or Gapmer is one or more of the sequences of Table 1. In some embodiments, the oligonucleotide therapeutic agent comprises one or more of SEQ ID NOs: 1-7. In other embodiments, the small molecule antagonist or inhibitor may bind to the Cdkn2a protein and inhibit its function.

By restoring the self-renewal ability of muscle satellite cells, the methods and compositions disclosed herein may be able to promote muscle regeneration and ameliorate the symptoms of myopathy. This could potentially lead to improved muscle function and a slowing or halting of disease progression. In some cases, the inhibitor of Cdkn2a may be administered in combination with one or more other therapeutic agents, such as SMN repletion therapies, to provide a more comprehensive treatment for myopathy.

In some aspects, the present disclosure provides a method of preventing premature aging of muscle satellite cells in a subject. This method may involve administering to the subject an effective amount of an inhibitor of Cdkn2a. As previously described, Cdkn2a is a cell cycle regulator that, when overexpressed or overactive, can lead to cellular senescence, a state of irreversible cell cycle arrest. In the context of muscle satellite cells, increased Cdkn2a activity can lead to premature aging of these cells, thereby impairing their ability to self-renew and contribute to muscle regeneration.

By inhibiting the expression or function of Cdkn2a, the methods disclosed herein may be able to prevent premature aging of muscle satellite cells. This could be achieved, for example, by using an oligonucleotide therapeutic agent or a small molecule antagonist or inhibitor that targets Cdkn2a. In some embodiments, the oligonucleotide therapeutic agent may be an RNAi molecule, an antisense oligonucleotide, a siRNA, a shRNA, or a Gapmer, each of which can bind to the Cdkn2a mRNA and prevent its translation into protein. In some embodiments, the siRNA and/or Gapmer is one or more of the sequences of Table 1. In some embodiments, the oligonucleotide therapeutic agent comprises one or more of SEQ ID NOs: 1-7. In other embodiments, the small molecule antagonist or inhibitor may bind to the Cdkn2a protein and inhibit its function.

By preventing premature aging of muscle satellite cells, the methods disclosed herein may be able to maintain the self-renewal ability of these cells, thereby promoting muscle regeneration and potentially ameliorating the symptoms of myopathy. This could potentially lead to improved muscle function and a slowing or halting of disease progression. In some cases, the inhibitor of Cdkn2a may be administered in combination with one or more other therapeutic agents, such as SMN repletion therapies, to provide a more comprehensive treatment for myopathy.

In some aspects, the methods disclosed herein may involve administering the inhibitor of Cdkn2a in combination with one or more SMN repletion therapies. SMN repletion therapies are treatments that aim to increase the levels of the survival motor neuron (SMN) protein, which is deficient in subjects with spinal muscular atrophy (SMA). These therapies may include, but are not limited to, drugs, gene therapies, or other treatments that increase the production or stability of the SMN protein.

In some cases, the combination of an inhibitor of Cdkn2a with one or more SMN repletion therapies may provide a synergistic effect, resulting in a more effective treatment or amelioration of myopathy. For example, the inhibitor of Cdkn2a may prevent premature aging of muscle satellite cells and restore their self-renewal potential, while the SMN repletion therapy may increase the levels of the SMN protein, thereby improving motor neuron function and potentially slowing or halting the progression of neuromuscular diseases such as SMA.

The specific type of SMN repletion therapy used in combination with the inhibitor of Cdkn2a may depend on various factors, including but not limited to, the specific type of myopathy being treated, the severity of the myopathy, the age, weight, and overall health of the subject, and the specific type of inhibitor of Cdkn2a being administered. In some embodiments, the SMN repletion therapy and the inhibitor of Cdkn2a may be administered concurrently. In other embodiments, the SMN repletion therapy may be administered before or after the administration of the inhibitor of Cdkn2a. The timing and sequence of administration may be determined based on the specific therapeutic goals and the individual characteristics of the subject.

In some aspects, the method of restoring self-renewal potential of muscle satellite cells in a subject may involve administering an effective amount of an inhibitor of Cdkn2a. The inhibitor of Cdkn2a may be administered in a variety of ways, including but not limited to, oral administration, intravenous injection, intramuscular injection, subcutaneous injection, intraperitoneal injection, intrathecal injection, or direct delivery to the target tissue. In some cases, the inhibitor of Cdkn2a may be administered intravenously. Intravenous administration may allow for rapid delivery of the inhibitor to the bloodstream, thereby facilitating its distribution to the muscle tissue where it can exert its effects on muscle satellite cells.

The inhibitor of Cdkn2a may act to prevent the premature aging of muscle satellite cells, thereby restoring their self-renewal potential. This may be particularly beneficial in the context of myopathies, where the ability of muscle satellite cells to self-renew and contribute to muscle regeneration may be compromised. By preventing premature aging of muscle satellite cells, the inhibitor of Cdkn2a may help to maintain the regenerative capacity of the muscle tissue, thereby potentially ameliorating the symptoms of myopathy and slowing or halting disease progression.

In some embodiments, the inhibitor of Cdkn2a may be an oligonucleotide therapeutic agent, such as an RNAi molecule, an antisense oligonucleotide, a siRNA, a shRNA, or a Gapmer. These oligonucleotide therapeutic agents may bind to the Cdkn2a mRNA and prevent its translation into protein, thereby inhibiting the function of Cdkn2a and preventing premature aging of muscle satellite cells. In some embodiments, the siRNA and/or Gapmer is one or more of the sequences of Table 1. In some embodiments, the inhibitor of Cdkn2a is an oligonucleotide therapeutic agent comprising one or more of SEQ ID NOs: 1-7. In other embodiments, the inhibitor of Cdkn2a may be a small molecule antagonist or inhibitor that binds to the Cdkn2a protein and inhibits its function. The specific type of inhibitor of Cdkn2a used may depend on various factors, including but not limited to, the specific type of myopathy being treated, the severity of the myopathy, the age, weight, and overall health of the subject, and the desired route of administration.

In some aspects, the inhibitor of Cdkn2a may be administered to a subject who is a human. The administration of the inhibitor of Cdkn2a to a human subject may be carried out in a variety of ways, depending on various factors such as the specific type of myopathy being treated, the severity of the myopathy, and the overall health of the subject. In some cases, the inhibitor of Cdkn2a may be administered intravenously. Intravenous administration allows for the rapid delivery of the inhibitor into the bloodstream, facilitating its distribution to the muscle tissue where it can exert its effects on muscle satellite cells. This route of administration may be particularly beneficial for treating or ameliorating myopathies that affect a large proportion of the body's muscle tissue, as it allows for the widespread distribution of the inhibitor. However, other routes of administration may also be used, depending on the specific circumstances and therapeutic goals.

In some embodiments, the method of treating or ameliorating myopathy in a subject may further comprise administering one or more SMN repletion therapies to the subject. SMN repletion therapies are treatments that aim to increase the levels of the survival motor neuron (SMN) protein, which is deficient in subjects with spinal muscular atrophy (SMA). These therapies may include, but are not limited to, drugs, gene therapies, or other treatments that increase the production or stability of the SMN protein.

In some cases, the combination of an inhibitor of Cdkn2a with one or more SMN repletion therapies may provide a synergistic effect, resulting in a more effective treatment or amelioration of myopathy. For example, the inhibitor of Cdkn2a may prevent premature aging of muscle satellite cells and restore their self-renewal potential, while the SMN repletion therapy may increase the levels of the SMN protein, thereby improving motor neuron function and potentially slowing or halting the progression of neuromuscular diseases such as SMA.

The specific type of SMN repletion therapy used in combination with the inhibitor of Cdkn2a may depend on various factors, including but not limited to, the specific type of myopathy being treated, the severity of the myopathy, the age, weight, and overall health of the subject, and the specific type of inhibitor of Cdkn2a being administered. In some embodiments, the SMN repletion therapy and the inhibitor of Cdkn2a may be administered concurrently. In other embodiments, the SMN repletion therapy may be administered before or after the administration of the inhibitor of Cdkn2a. The timing and sequence of administration may be determined based on the specific therapeutic goals and the individual characteristics of the subject.

Accordingly, in some aspects the present disclosure provides a method of treating or ameliorating myopathy in a subject in need thereof comprising the step of administering to the subject an effective amount of an inhibitor of Cdkn2a. In some embodiments, the myopathy is a neuromuscular disease. In some embodiments, the myopathy is sarcopenia. In some embodiments, the myopathy is spinal muscular atrophy (SMA). In some embodiments, the myopathy is Duchenne Muscular Dystrophy (DMD). In some embodiments, the inhibitor of Cdkn2a is one or more oligonucleotide therapeutic agents. In some embodiments, the inhibitor of Cdkn2a is one or more of RNAi and antisense oligonucleotides. In some embodiments, the inhibitor of Cdkn2a is one or more of siRNAs, shRNAs and Gapmers. In some embodiments, the siRNA and/or Gapmer is one or more of the sequences of Table 1. In some embodiments, the inhibitor of Cdkn2a is an oligonucleotide therapeutic agent comprising one or more of SEQ ID NOs: 1-7. In some embodiments, the inhibitor of Cdkn2a is a small molecule antagonists/inhibitor. In some such embodiments, the inhibitor of Cdkn2a is a small molecule antagonist/inhibitor selected from one or more of 4-(Methylnitrosamino)-1-(3-pyridyl)-1-butanone, 5-Aza-2′deoxycytidine, 5-fluorouracil, 5-Methylcytosine, 5-metylthioadenosine, 7,12-dimethylbenz (a) anthracene, acnu, Aflatoxin B1, Agar, and Agarose. In some embodiments, the Gapmer is the antisense morpholino Gapmer (Qiagen, Inc.). In some embodiments, the shRNA is delivered via vector. In some embodiments, the shRNA is delivered via adeno-associated virus vector. In some embodiments, the shRNA is delivered via AAV9 vector. In some embodiments, the inhibitor of Cdkn2a is effective to restore the ability of self-renewal in one or more muscle satellite cells. In some embodiments, the inhibitor of Cdkn2a is effective to prevent premature aging in one or more muscle satellite cells. In some embodiments, the method of treating or ameliorating myopathy in a subject further comprises the step of administering one or more SMN repletion therapies.

According to some aspects, the present disclosure provides a method of preventing premature aging of muscle satellite cells in a subject comprising the step of administering to the subject an effective amount of an inhibitor of Cdkn2a. In some embodiments, the inhibitor of Cdkn2a is one or more oligonucleotide therapeutic agents. In some embodiments, the inhibitor of Cdkn2a is one or more of RNAi and antisense oligonucleotides. In some embodiments, the inhibitor of Cdkn2a is one or more of siRNAs, shRNAs and Gapmers. In some embodiments, the Gapmer is the antisense morpholino Gapmer (Qiagen, Inc.). In some embodiments, the siRNA and/or Gapmer is one or more of the sequences of Table 1. In some embodiments, the inhibitor of Cdkn2a is an oligonucleotide therapeutic agent comprising one or more of SEQ ID NOs: 1-7. In some embodiments, the shRNA is an AAV9-shRNA. In some embodiments, the inhibitor of Cdkn2a is a small molecule antagonists/inhibitor. In some such embodiments, the inhibitor of Cdkn2a is a small molecule antagonists/inhibitor selected from one or more of 4-(Methylnitrosamino)-1-(3-pyridyl)-1-butanone, 5-Aza-2′deoxycytidine, 5-fluorouracil, 5-Methylcytosine, 5-metylthioadenosine, 7,12-dimethylbenz (a) anthracene, acnu, Aflatoxin B1, Agar, and Agarose. In some embodiments, the inhibitor of Cdkn2a is effective to treat or ameliorate a myopathy. In some embodiments, the inhibitor of Cdkn2a is effective to treat or ameliorate one or more of a neuromuscular disease or of sarcopenia. In some embodiments, the inhibitor of Cdkn2a is effective to treat or ameliorate one or more of spinal muscular atrophy (SMA) and Duchenne Muscular Dystrophy (DMD).

According to some aspects, the present disclosure provides a method of restoring self-renewal potential of muscle satellite cells in a subject comprising the step of administering to the subject an effective amount of an inhibitor of Cdkn2a. In some embodiments, the inhibitor of Cdkn2a is one or more oligonucleotide therapeutic agents. In some embodiments, the inhibitor of Cdkn2a is one or more of RNAi and antisense oligonucleotides. In some embodiments, the inhibitor of Cdkn2a is one or more of siRNAs, shRNAs and Gapmers. In some embodiments, the Gapmer is the antisense morpholino Gapmer (Qiagen, Inc.). In some embodiments, the siRNA and/or Gapmer is one or more of the sequences of Table 1. In some embodiments, the inhibitor of Cdkn2a is an oligonucleotide therapeutic agent comprising one or more of SEQ ID NOs: 1-7. In some embodiments, the shRNA is an AAV9-shRNA. In some embodiments, the inhibitor of Cdkn2a is a small molecule antagonist/inhibitor. In some such embodiments, the inhibitor of Cdkn2a is a small molecule antagonist/inhibitor selected from one or more of 4-(Methylnitrosamino)-1-(3-pyridyl)-1-butanone, 5-Aza-2′deoxycytidine, 5-fluorouracil, 5-Methylcytosine, 5-metylthioadenosine, 7,12-dimethylbenz (a) anthracene, acnu, Aflatoxin B1, Agar, and Agarose. In some embodiments, the restoring of self-renewal by administration of inhibitor of Cdkn2a is effective to treat or ameliorate a myopathy. In some embodiments, the restoring of self-renewal by administration of inhibitor of Cdkn2a is effective to treat or ameliorate one or more of a neuromuscular disease or of sarcopenia. In some embodiments, the restoring of self-renewal by administration of inhibitor of Cdkn2a is effective to treat or ameliorate one or more of spinal muscular atrophy (SMA) and Duchenne Muscular Dystrophy (DMD).

According to some aspects, the present disclosure provides a composition for use in preventing premature aging or restoring self-renewal of muscle satellite cells in a subject, the composition comprising an inhibitor of Cdkn2a. In some embodiments, the inhibitor of Cdkn2a is one or more oligonucleotide therapeutic agents. In some embodiments, the inhibitor of Cdkn2a is one or more of RNAi and antisense oligonucleotides. In some embodiments, the inhibitor of Cdkn2a is one or more of siRNAs, shRNAs and Gapmers. In some embodiments, the Gapmer is the antisense morpholino Gapmer (Qiagen, Inc.). In some embodiments, the siRNA and/or Gapmer is one or more of the sequences of Table 1. In some embodiments, the inhibitor of Cdkn2a is an oligonucleotide therapeutic agent comprising one or more of SEQ ID NOs: 1-7. In some embodiments, the shRNA is an AAV9-shRNA. In some embodiments, the inhibitor of Cdkn2a is a small molecule antagonist/inhibitor. In some such embodiments, the inhibitor of Cdkn2a is a small molecule antagonist/inhibitor selected from one or more of 4-(Methylnitrosamino)-1-(3-pyridyl)-1-butanone, 5-Aza-2′deoxycytidine, 5-fluorouracil, 5-Methylcytosine, 5-metylthioadenosine, 7,12-dimethylbenz (a) anthracene, acnu, Aflatoxin B1, Agar, and Agarose. In some embodiments, the composition further comprises one or more SMN repletion therapies.

As used herein, the terms “treat,” “treating,” “treatment” and grammatical variations thereof mean subjecting an individual subject to a protocol, regimen, process or remedy, in which it is desired to obtain a physiologic response or outcome in that subject, e.g., a patient. In particular, the methods and compositions of the present disclosure may be used to slow the development of disease symptoms or delay the onset of the disease or condition, or halt the progression of disease development. However, because not every treated subject may respond to a particular treatment protocol, regimen, process or remedy, treating does not require that the desired physiologic response or outcome be achieved in each and every subject or subject population, e.g., patient population. Accordingly, a given subject or subject population, e.g., patient population, may fail to respond or respond inadequately to treatment.

As used herein, the terms “ameliorate”, “ameliorating” and grammatical variations thereof mean to decrease the severity of the symptoms of a disease in a subject.

In the present disclosure, an “effective amount” of a therapeutic is an amount of such therapeutic that is sufficient to effect beneficial or desired results as described herein when administered to a subject. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of the subject, and like factors well known in the arts of, e.g., medicine and veterinary medicine. In general, a suitable dose of a therapeutic according to the disclosure will be that amount of the agent, which is the lowest dose effective to produce the desired effect with no or minimal side effects. The effective dose of a therapeutic according to the present disclosure may be administered as a single dose, or two, three, four, five, six or more sub-doses, administered separately at appropriate intervals throughout the treatment course (e.g., not necessarily on the same day).

As used herein, a “subject” is a mammal, preferably, a human. In addition to humans, categories of mammals within the scope of the present disclosure include, for example, agricultural animals, veterinary animals, laboratory animals, etc. Some examples of agricultural animals include cows, pigs, horses, goats, etc. Some examples of veterinary animals include dogs, cats, etc. Some examples of laboratory animals include primates, rats, mice, rabbits, guinea pigs, etc. In some embodiments of the present disclosure, the phrase “a subject” means a subject having a myopathy, such as, e.g., a neuromuscular disease, sarcopenia, spinal muscular atrophy (SMA), or Duchenne Muscular Dystrophy (DMD).

The terminology used herein is for the purpose of describing particular embodiments only and is not intended to be limiting. As used in the specification and the appended claims, the singular forms “a,” “an,” and “the” include plural referents unless the context clearly dictates otherwise.

The following examples are provided to further illustrate the compositions and methods of the present disclosure. These examples are illustrative only and are not intended to limit the scope of the disclosure in any way.

### EXAMPLES

### Example 1

Spinal muscular atrophy (SMA) is an oft-fatal pediatric onset neuromuscular disorder caused by mutations in the SMN1 gene and low level of the SMN protein. A paralogue, SMN2, only exists in humans but cannot compensate for loss of SMN1 owing to the low levels of protein expressed by SMN2. The SMN protein is important in both neuronal and muscle tissues. Indeed, our findings suggest that selective depletion of SMN protein in muscle is profoundly damaging and triggers disease. Restoration of the protein in our model reverses muscle pathology. Yet, the precise origin of the muscle pathology we have observed is unclear. Defects in muscle stem cells (satellite cells) could potentially contribute to the pathology. In the present study, we have examined this possibility.

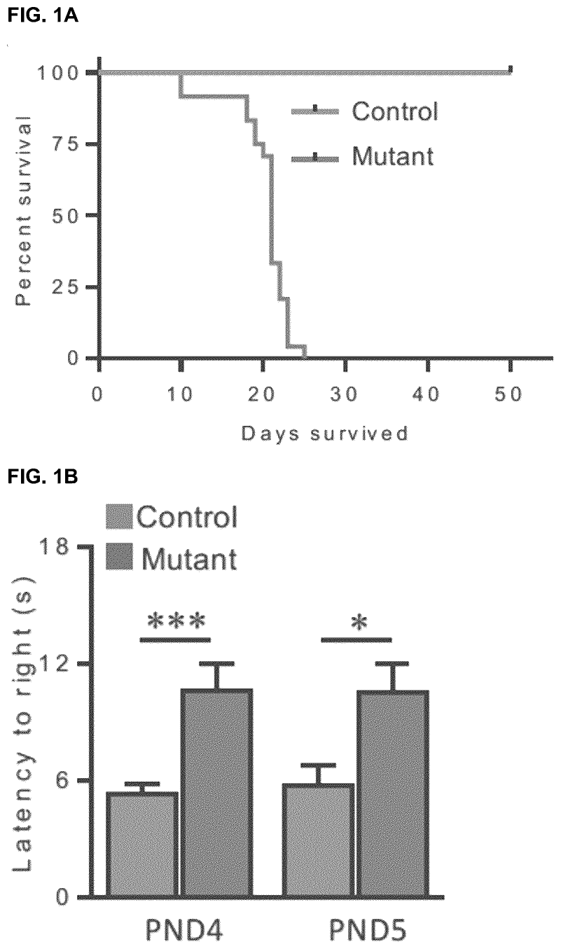

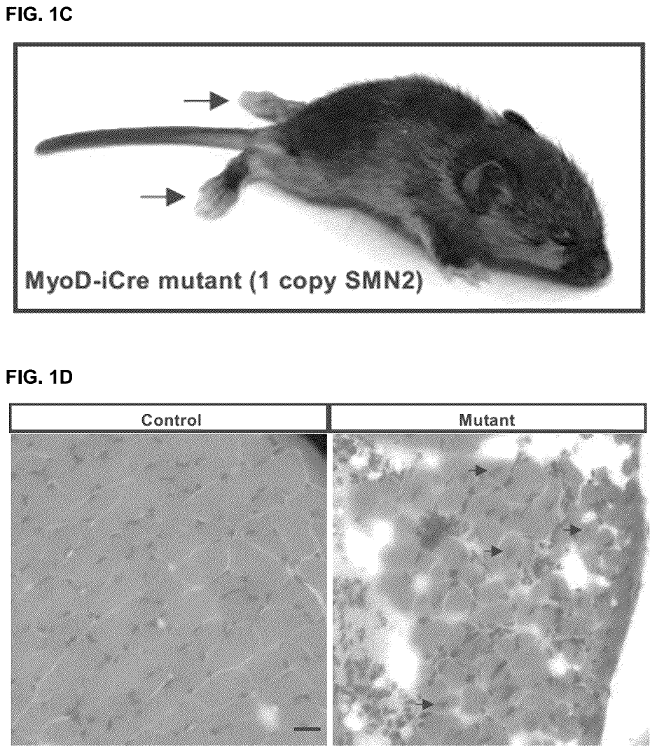

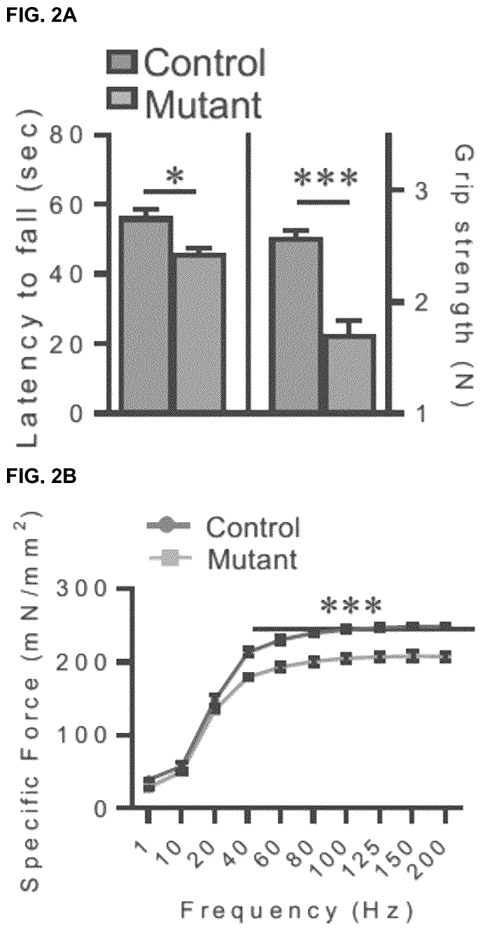

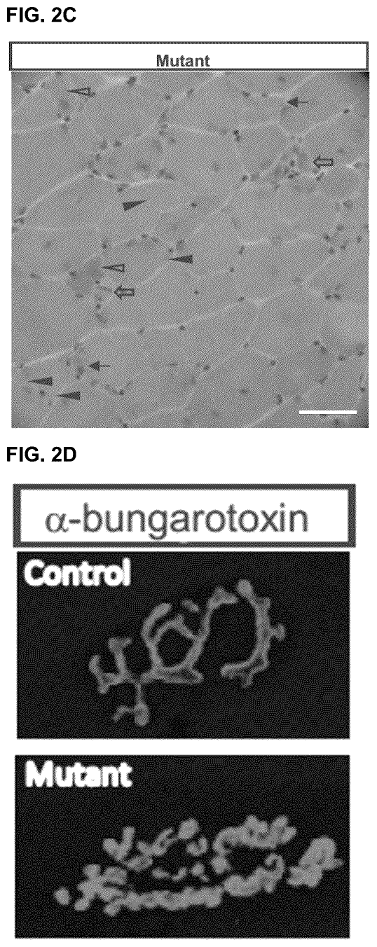

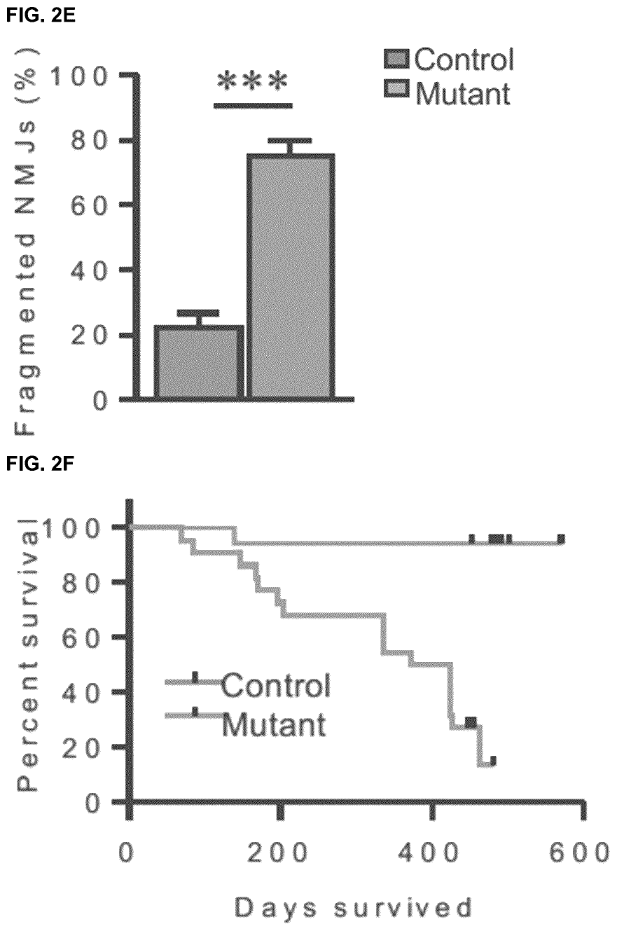

SMN is a ubiquitously expressed protein. Yet, SMN paucity triggers predominantly neuromuscular dysfunction. Skeletal muscle pathology in SMA has traditionally been assumed to result from a primary defect in spinal motor neurons. However, it has been shown that low SMN in skeletal muscle is sufficient to trigger muscle pathology (Kim, J-K et al, PMID: 32039917). Model mice in which SMN is selectively reduced in skeletal muscle exhibit muscle pathology and an overt phenotype. The severity of disease in the mutants depends on absolute levels of SMN. Model mice whose skeletal muscle expresses only one copy of the SMN2 gene are very severely affected. Mutants whose muscle expresses incrementally more SMN (from two SMN2 copies) are less severely affected (FIGS. 1A-1D). Nevertheless, they suffer from a late-onset myopathy and succumb to disease by ˜13 months of age. (FIGS. 2A-2F)

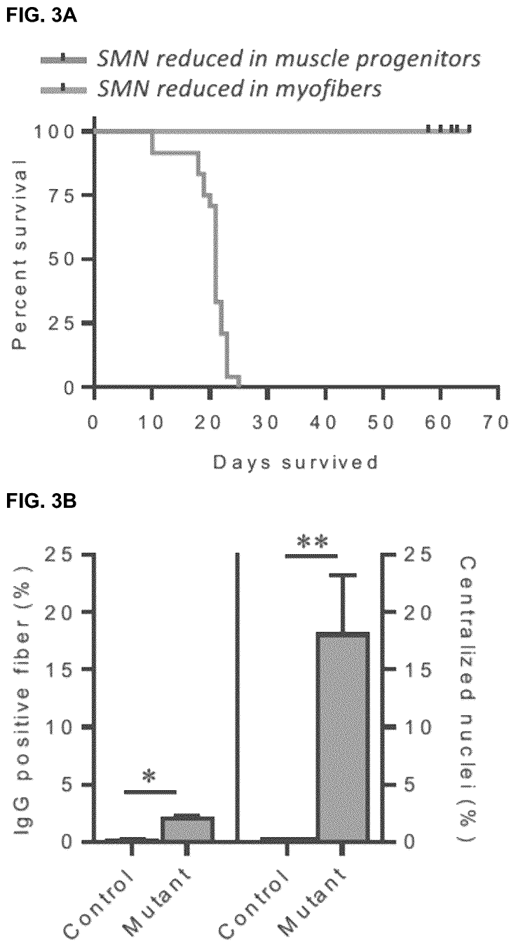

To ascertain whether low SMN evokes muscle pathology by affecting mature myofibers, muscle progenitors or both, we selectively depleted the protein in myofibers. Such mutants were more severely affected than mutants in which SMN was depleted in myofibers and muscle progenitors (satellite cells) (FIG. 3A). Moreover, we found that a disproportionately high number of myofibers exhibited evidence of dysfunction of satellite cells relative to degeneration of the fibers (FIG. 3B). This strongly suggests that muscle pathology in SMA must arise at least in part in muscle satellite cells.

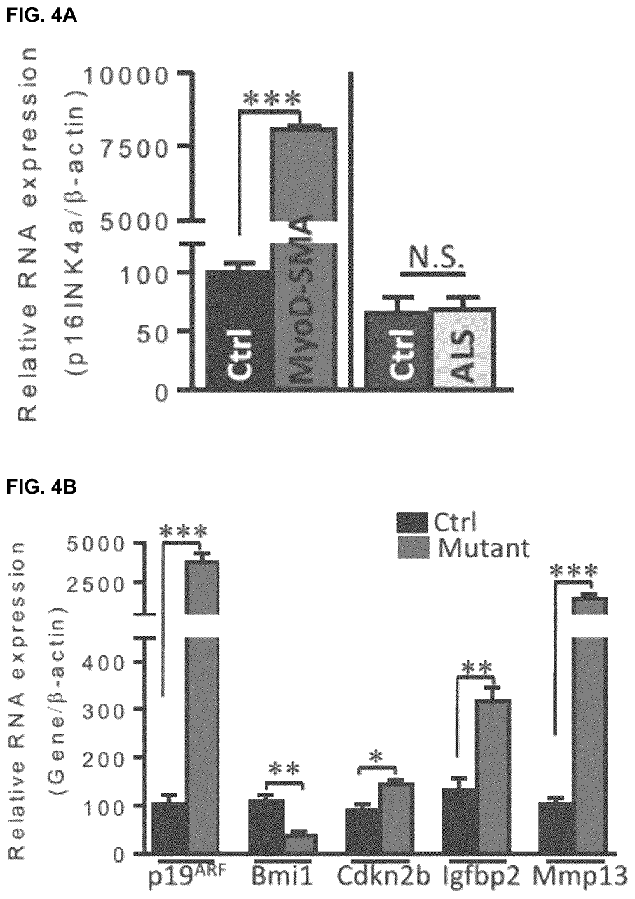

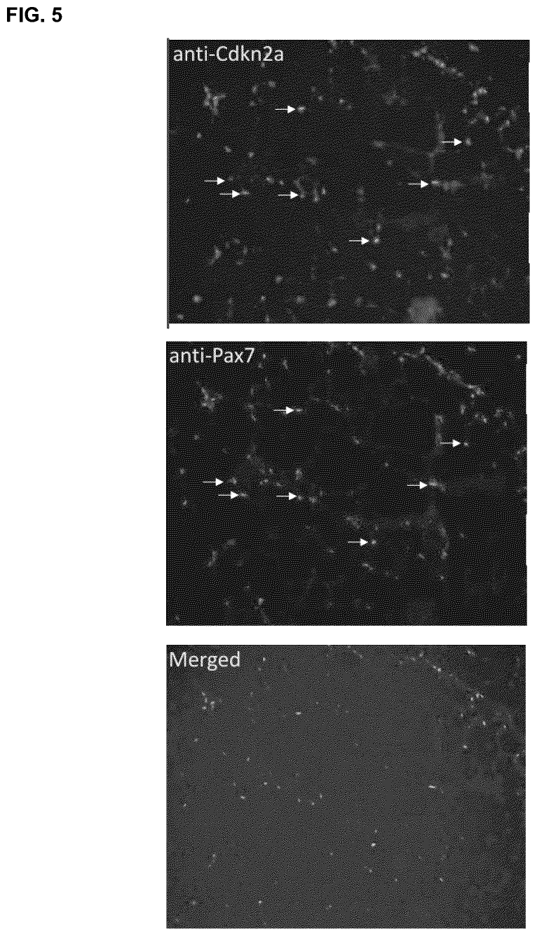

To investigate the molecular basis of muscle defects in SMA, we carried out RNA-Seq experiments on muscle of mutant mice. Our analysis revealed a profound perturbation of Cdkn2a, a cell-cycle regulator that controls the ability of muscle satellite cells to self-renew and return to quiescence. Upregulation of Cdkn2a activates satellite cells but eventually triggers senescence in them. In such a state, the satellite cells are unable to self-renew and become exhausted during muscle regeneration. Depletion of these cells prevents muscle regeneration and eventually causes loss of the muscle and death of the organism. Our analysis revealed a >6-fold increase of Cdkn2a in SMA muscle. Q-PCR confirmed this to be the case (FIG. 4A). Moreover, such an increase was not seen in muscle of amyotrophic lateral sclerosis (ALS) mice. Finally, genes associated with Cdkn2a were also perturbed in SMA muscle (FIG. 4B). To determine precisely the source of the increased expression of Cdkn2a, immunohistochemistry was carried out on muscle of SMA mutants. We demonstrated that muscle satellite cells were the source of the increased Cdkn2a expression (FIG. 5).

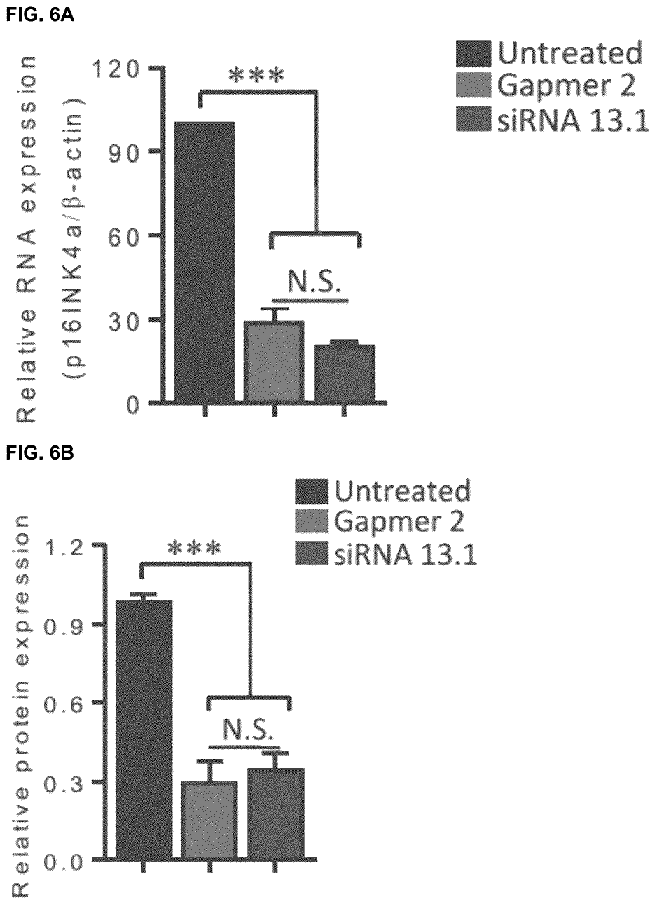

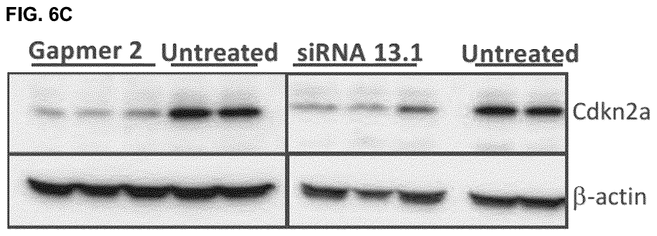

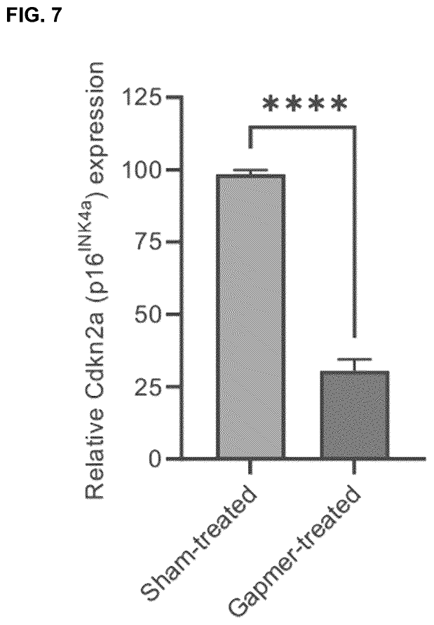

In the context of aging (sarcopenia), suppression of Cdkn2a in progeric satellite cells reinvigorates them, allowing resumption of self-renewal. We posit that doing so in the context of SMA will restore function to satellite cells and combat muscle pathology in the disease. To test whether Cdkn2a can be efficiently knocked down, we generated siRNAs and Gapmers against it. These were found to robustly knockdown Cdkn2a RNA as well as protein (FIGS. 6A-6C, see Table 1). To determine if such knockdown could be effected in vivo, a mouse model of muscular dystrophy was employed. These mice also express high levels of Cdkn2a in muscle. Cdkn2a expression is significantly reduced in such mutants injected with a Gapmer against the gene (FIG. 7). We posit that suppressing Cdkn2a expression in SMA will combat muscle disease and could be used in combination with currently available SMN repletion treatments as an adjunct therapy. Perturbed Cdkn2a regulation in other muscle diseases means suppressing its activity in satellite cells could become more generally relevant for the larger family of muscle diseases including DMD.

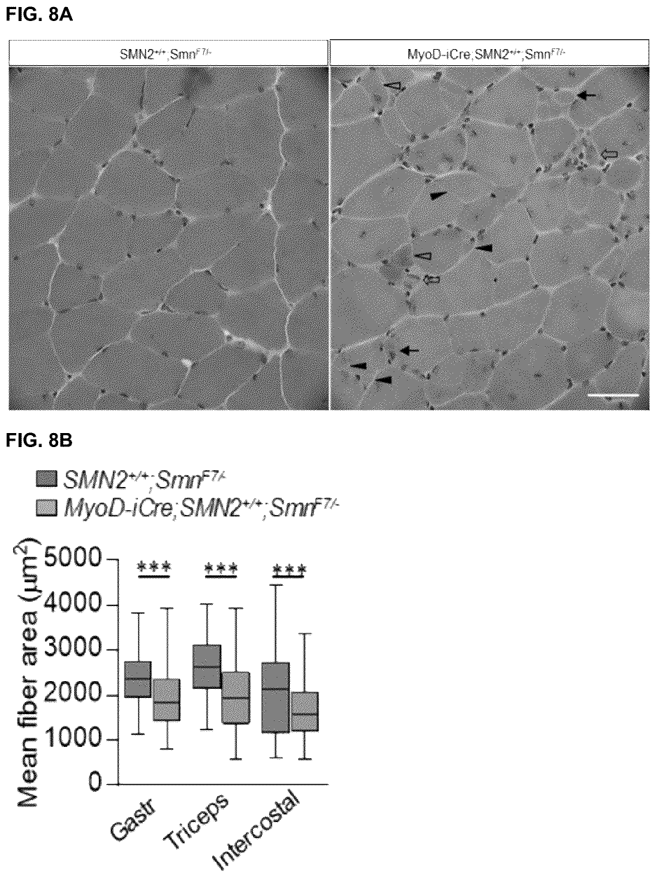

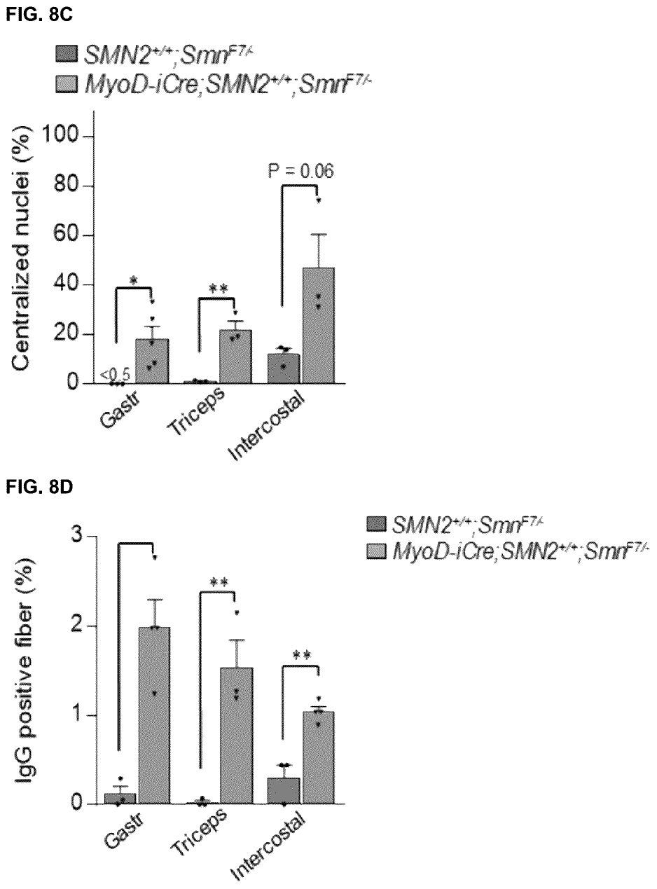

Disclosed herein is a MyoD-iCre driver to selectively deplete the Smn protein by removing a flexon in muscles of model mice with either 1 or 2 copies of SMN2. The mutants we generated represent SMA models in which the CNS but not muscle is restored for SMN. We found that muscle deprived of SMN was profoundly damaged. Although a disease phenotype was not immediately obvious, persistent low levels of the protein eventually resulted in muscle fiber defects, neuromuscular junction abnormalities, compromised motor performance, and premature death. Importantly, restoring SMN after the onset of muscle pathology reversed disease. See FIGS. 8A-8D.

### Results

### Elevated Number of Satellite Cells in MyoD-Cre SMA Mutant Mice

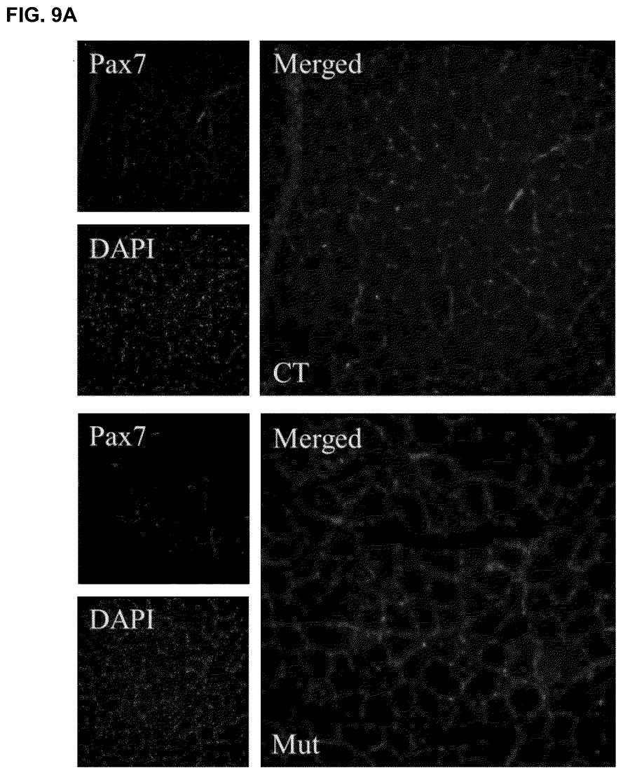

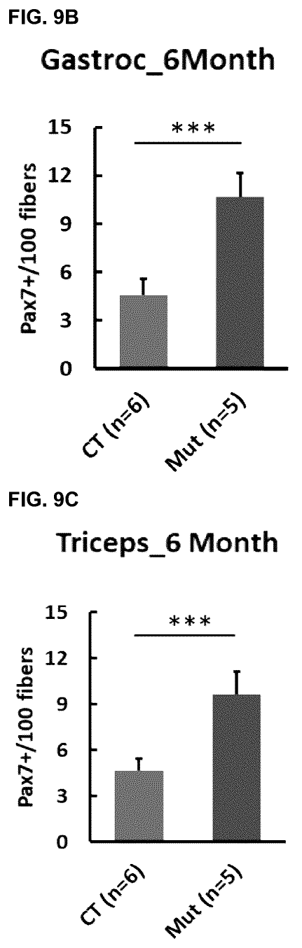

Satellite cells are myogenic stem cells involved in repair, maintenance and remodeling of muscle fibers. Therefore, our first objective was to quantify the number of satellite cells in the gastrocnemius and triceps muscles selectively depleted of Smn protein. Pax7 is a transcription factor expressed at high level in the satellite cells, hence used as a marker for identification of the myogenic stem cells in muscle sections. See FIGS. 9A-9C.

### Cell Cycle-Specific Genes are Upregulated in MyoD-Cre SMA Mutant Mice

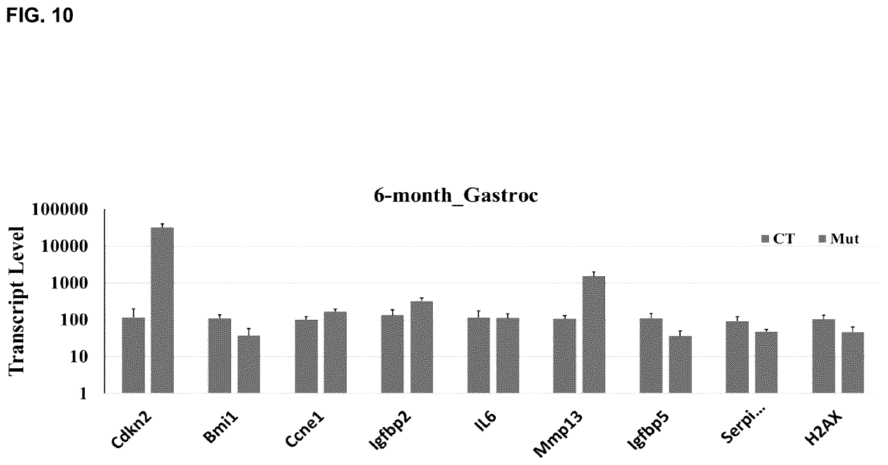

We performed RNA-Seq analysis to investigate the molecular basis of muscle pathology in the SMN-depleted muscle. Detailed analysis of the RNA-seq data showed upregulation of 116 transcripts and downregulation of 21 transcripts in the MyoD mutant mice. We confirmed the transcript levels of selected genes (high p-value) by qPCR and found an upregulation of cell cycle regulators in the muscle of 6-month mice. See FIG. 10.

### Enhanced Satellite Cell Numbers and Upregulation of Cell Cycle-Specific Genes are a Late Onset Event

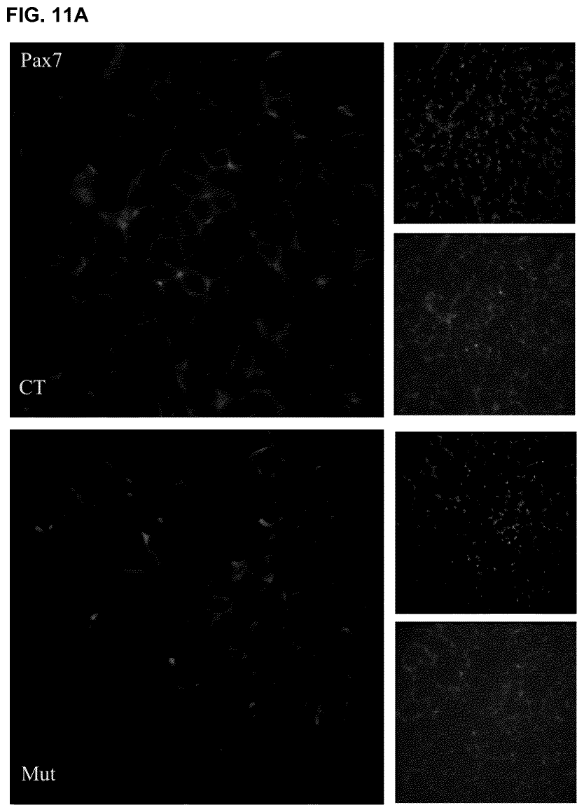

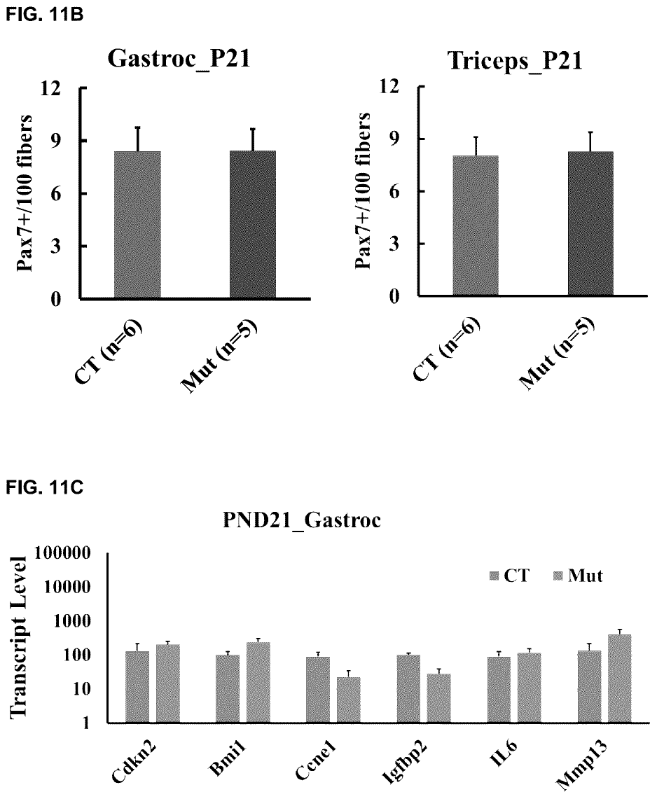

As disclosed herein, enhanced satellite cell numbers and upregulation of cell cycle-specific genes are shown to be a late onset event. See FIGS. 11A-11C.

### Enhanced Satellite Cell Number and Upregulation of Cell Cycle Regulator Genes are Generic Property of Muscle Pathology

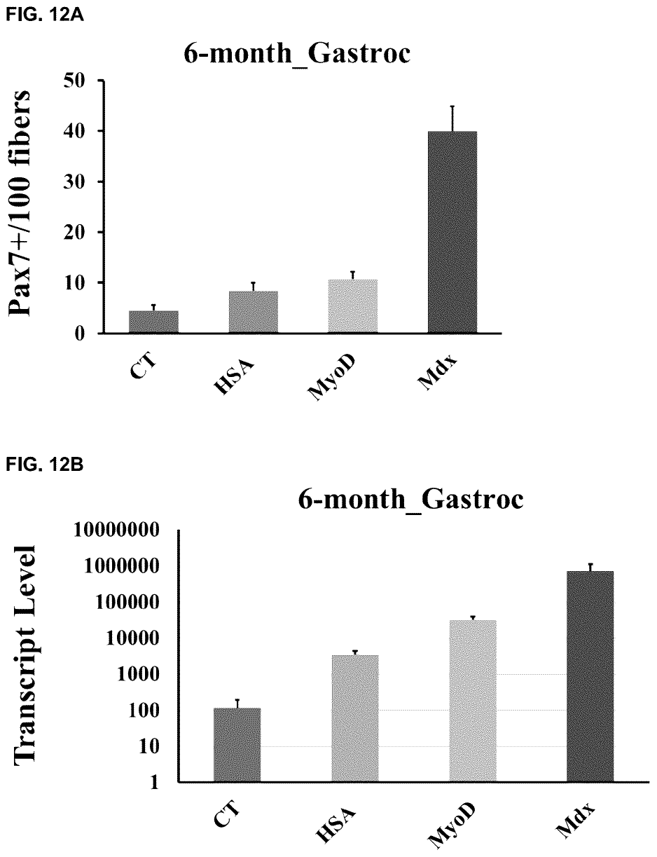

To check if differences in satellite cells and transcript level observed are specific to MyoD mut mice or are general properties of dystrophic muscle, we quantified the number of satellite cells and the transcript level of Cdkn2 gene in the muscles derived from different SMA mice models as well as from DMD-Mdx mice. See FIGS. 12A-12B.

### Does Knock-Down of Cell Cycle Regulator Genes Reverse the Satellite Cell Number and Reverse the Disease Pathology?

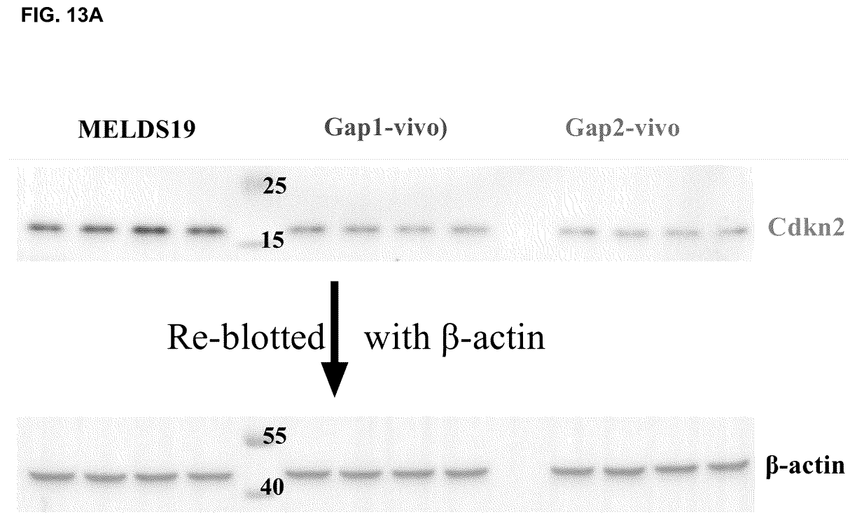

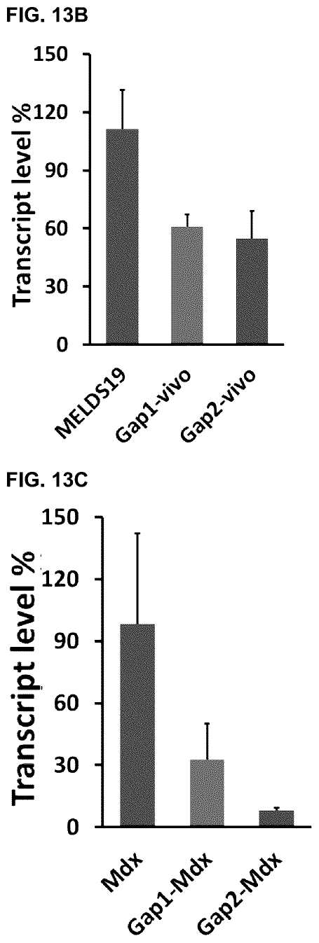

We designed and checked the efficacy of two vivo-gapmers against the candidate gene and ascertained their efficacy in cells and Mdx mice. The designed gapmers showed knockdown of the candidate gene in cell and Mdx model mouse. Our next aim is to check the knockdown efficacy of these gapmers in MyoD-SMA mouse model and their role in the reversal of muscle pathology. See FIGS. 13A-13C.

### Myofiber-Specific Depletion of SMN Results in Reduced Disease Severity

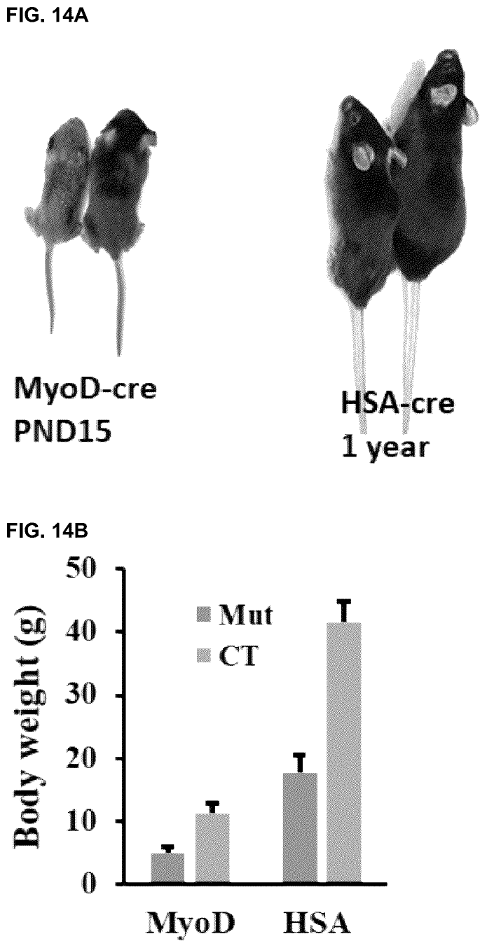

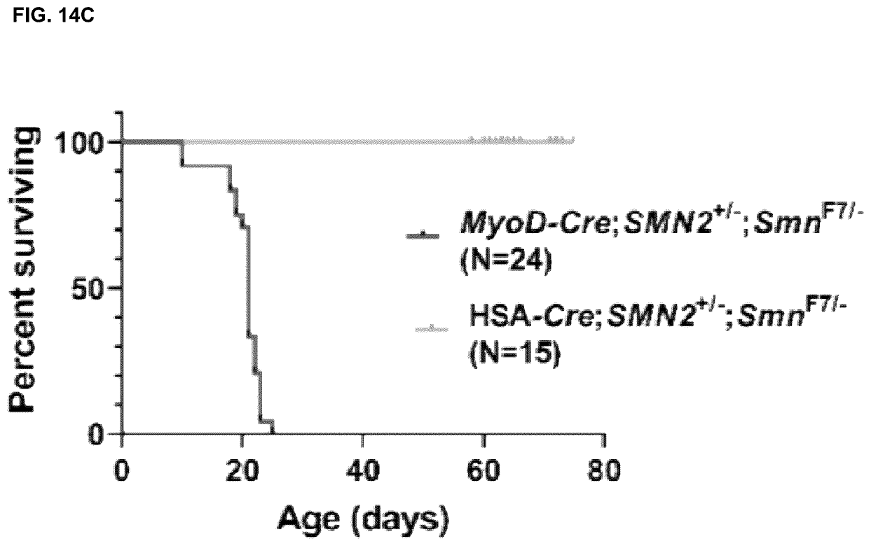

As disclosed herein, myofiber-specific depletion of SMN results in reduced disease severity. See FIGS. 14A-14C.

### Test of Gapmers Against the Cdkn2a Locus in Model Mice

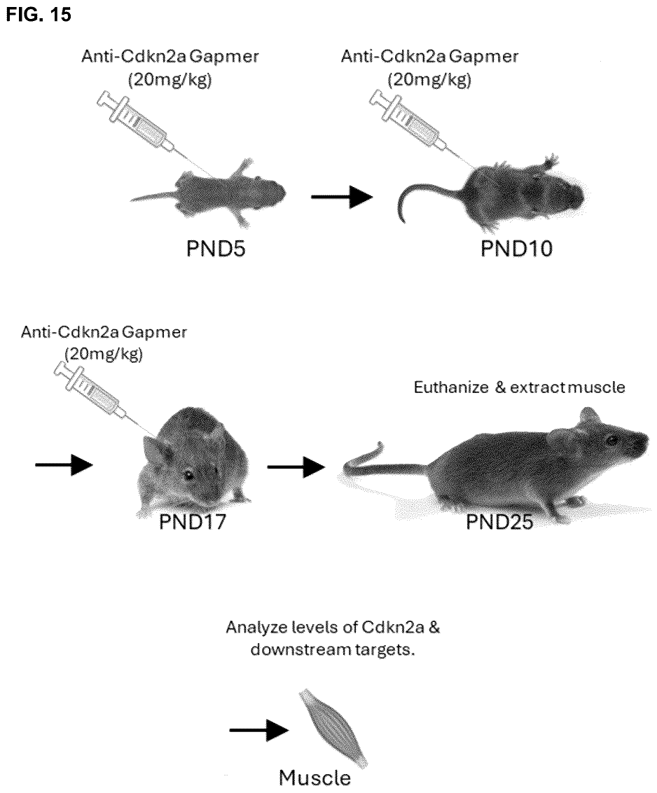

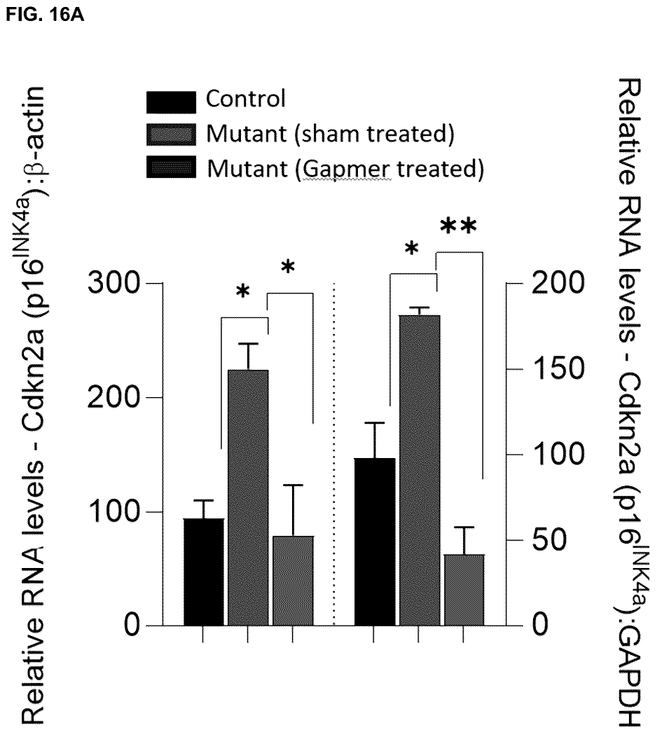

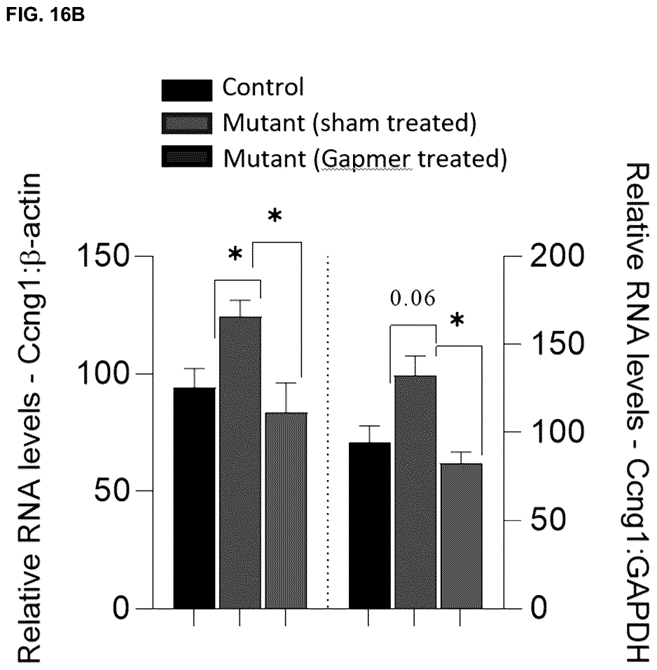

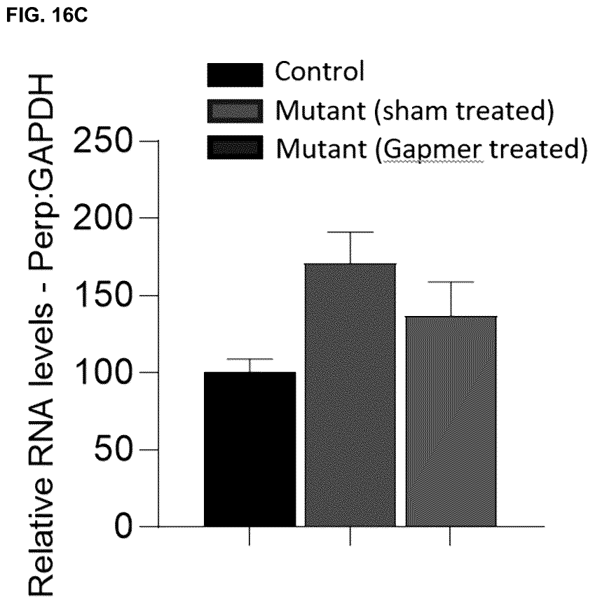

To provide a proof-of-concept of our ability to restore expression from the senescence-associated Cdkn2a locus as a means to prevent myopathy, we administered a Gapmer directed against the locus to a mouse model of spinal muscular atrophy selectively depleted of the SMN protein in muscle tissue. Following three administrations at PND5, PND10 and PND17, mutants and relevant controls were euthanized at PND25 to harvest muscle tissue (FIG. 15). Levels of Cdkn2a and its downstream targets were analyzed to determine ability to restore deranged expression of these loci in mutant mice (FIG. 16A). Delivery of Gapmer was found to restore expression of Cdkn2a to wild-type levels. Cong1 and Perp, two downstream targets of Cdkn2a were also reduced compared to expression of these genes in sham-treated mutants (FIGS. 16B-16C). These in vivo tests of an agent capable of normalizing Cdkn2a levels raise optimism that the locus may be efficiently targeted in models of muscle disease to combat myopathy.

Knockdown of Cdkn2a (p16^{INK4a}) Ameliorates Muscle Defects

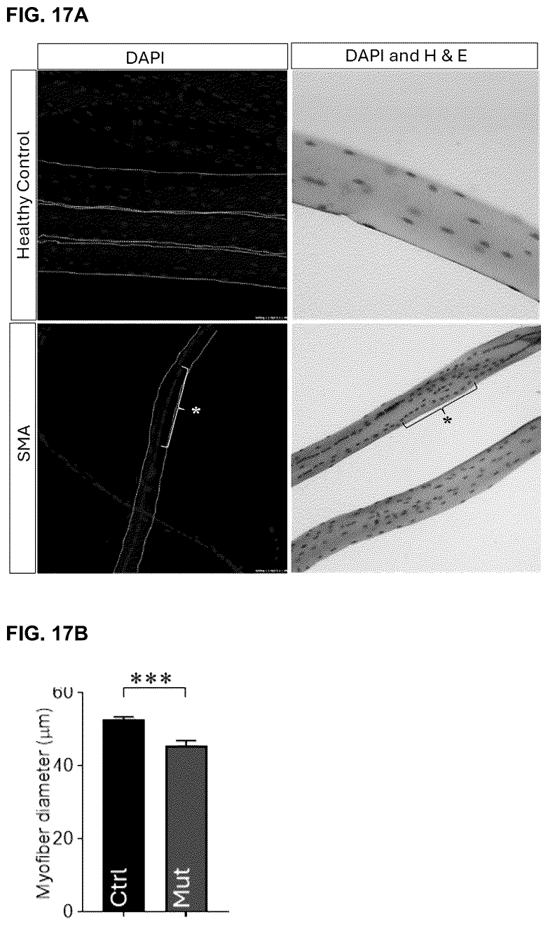

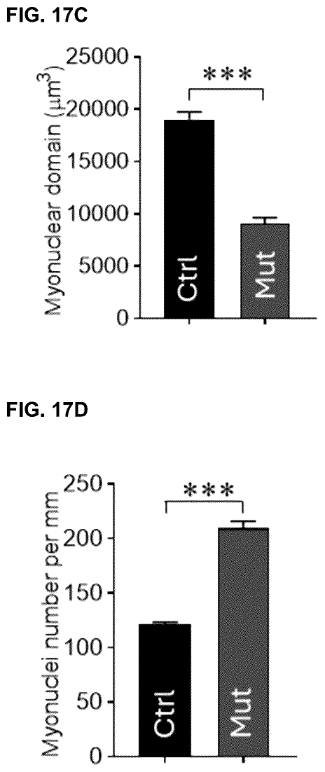

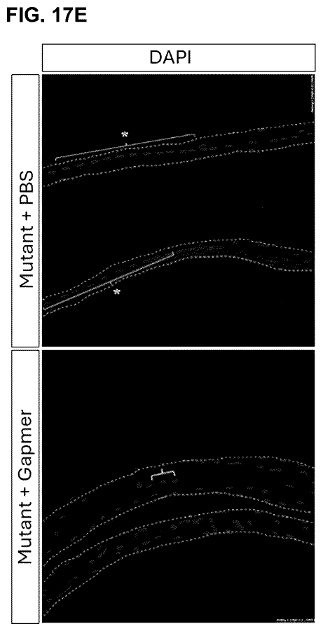

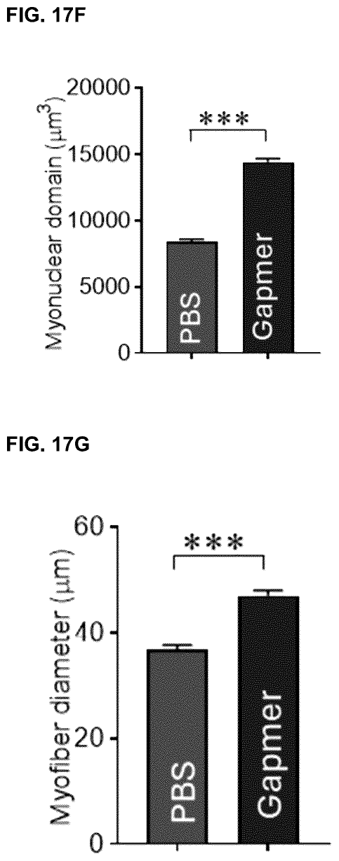

As disclosed herein, knockdown of p16^{INK4a} ameliorates muscle defects in SMA model mice. Compared to controls, mice treated with an anti-p16^{INK4a} Gapmer had fewer central nuclei and enhanced myonuclear domain and myofiber diameter. See FIGS. 17A-17G.

### CONCLUSION

Selective depletion of SMN protein in muscle leads to increase in the number of satellite cells in the skeletal muscles of symptomatic two-copy SMN2 MyoD-cre SMA mice. In contrast, no reduction in satellite cells was seen in pre-symptomatic two-copy SMN2 mice. Our RNA-seq data indicates dysregulation of several genes involved in senescence. We have also found that selective loss of SMN in muscle perturbs expression of several genes involved in cell-cycle regulation. Our results show the possibility that modulating a pathway associated with these genes, which is known to be involved in muscle dysfunction, could mitigate the SMA phenotype. Our findings are also suggestive of a heretofore-unreported link between SMN and the cell-cycle in muscle tissue.

### REFERENCES

1. Kim J K, Jha N N, Feng Z, Faleiro M R, Chiriboga C A, Wei-Lapierre L, Dirksen R T, Ko C-P, Monani U R Muscle-specific SMN reduction reveals motor neuron-independent disease in spinal muscular atrophy models. J Clin Invest. 2020 Mar. 2; 130 (3): 1271-1287.
2. Sophie Nicole, Benedicte Desforges, Gaelle Millet, Jeanne Lesbordes, Carmen Cifuentes-Diaz, Dora Vertes, My Linh Cao, Fabienne De Backer, Laetitia Languille, Natacha Roblot, Vandana Joshi, Jean-Marie Gillis, Judith Melki; Intact satellite cells lead to remarkable protection against Smn gene defect in differentiated skeletal muscle. J Cell Biol 12 May 2003; 161 (3): 571-582.
3. Hayhurst M, Wagner A K, Cerletti M, Wagers A J, Rubin L L. A cell-autonomous defect in skeletal muscle satellite cells expressing low levels of survival of motor neuron protein. Dev Biol. 2012 Aug. 15; 368 (2): 323-34.
4. Sousa-Victor P, Gutarra S, García-Prat L, Rodriguez-Ubreva J, Ortet L, Ruiz-Bonilla V, Jardí M, Ballestar E, González S, Serrano A L, Perdiguero E, Muñoz-Cánoves P. Geriatric muscle stem cells switch reversible quiescence into senescence. Nature. 2014 Feb. 20; 506 (7488): 316-21.

All documents cited in this application are hereby incorporated by reference as if recited in full herein. In the event of a conflict between the teachings of this application and those of the incorporated documents, the teachings of this application control.

Although illustrative embodiments of the present disclosure have been described herein, it should be understood that the disclosure is not limited to those described, and that various other changes or modifications may be made by one skilled in the art without departing from the scope or spirit of the disclosure.

What is claimed is:

1. A method of treating or ameliorating myopathy in a subject in need thereof comprising the step of administering to the subject an effective amount of an inhibitor of Cdkn2a.

2. The method of claim 1, wherein the myopathy is a neuromuscular disease.

3. The method of claim 1, wherein the myopathy is sarcopenia.

4. The method of claim 1, wherein the myopathy is spinal muscular atrophy (SMA).

5. The method of claim 1, wherein the myopathy is Duchenne Muscular Dystrophy (DMD).

6. The method of claim 1, wherein the inhibitor of Cdkn2a is one or more oligonucleotide therapeutic agents.

7. The method of claim 6, wherein the inhibitor of Cdkn2a is one or more of RNAi and antisense oligonucleotides.

8. The method of claim 7, wherein the inhibitor of Cdkn2a is one or more of siRNAs, shRNAs and Gapmers.

9. The method of claim 1, wherein the inhibitor of Cdkn2a is a small molecule antagonist/inhibitor.

10. The method of claim 1, wherein the inhibitor of Cdkn2a is effective to restore the ability of self-renewal in one or more muscle satellite cells.

11. The method of claim 1, wherein the inhibitor of Cdkn2a is effective to prevent premature aging in one or more muscle satellite cells.

12. The method of claim 1, further comprising the step of administering one or more SMN repletion therapies.

13. The method of claim 1, wherein the inhibitor of Cdkn2a is an oligonucleotide therapeutic agent comprising one or more of SEQ ID NOs: 1-7.

14. A method of preventing premature aging of muscle satellite cells in a subject comprising the step of administering to the subject an effective amount of an inhibitor of Cdkn2a.

15. The method of claim 14, wherein the inhibitor of Cdkn2a is one or more oligonucleotide therapeutic agents.

16. The method of claim 15, wherein the inhibitor of Cdkn2a is one or more of RNAi and antisense oligonucleotides.

17. The method of claim 16, wherein the inhibitor of Cdkn2a is one or more of siRNAs, shRNAs and Gapmers.

18. The method of claim 14, wherein the inhibitor of Cdkn2a is a small molecule antagonist/inhibitor.

19. The method of claim 14, wherein the inhibitor of Cdkn2a is effective to treat or ameliorate a myopathy.

20. The method of claim 14, wherein the inhibitor of Cdkn2a is effective to treat or ameliorate one or more of a neuromuscular disease and sarcopenia.

21. The method of claim 14, wherein the inhibitor of Cdkn2a is effective to treat or ameliorate one or more of spinal muscular atrophy (SMA) and Duchenne Muscular Dystrophy (DMD).

22. The method of claim 14, wherein the inhibitor of Cdkn2a is an oligonucleotide therapeutic agent comprising one or more of SEQ ID NOs: 1-7.

23. A method of restoring self-renewal potential of muscle satellite cells in a subject comprising the step of administering to the subject an effective amount of an inhibitor of Cdkn2a.

24. The method of claim 23, wherein the inhibitor of Cdkn2a is one or more oligonucleotide therapeutic agents.

25. The method of claim 24, wherein the inhibitor of Cdkn2a is one or more of RNAi and antisense oligonucleotides.

26. The method of claim 25, wherein the inhibitor of Cdkn2a is one or more of siRNAs, shRNAs and Gapmers.

27. The method of claim 23, wherein the inhibitor of Cdkn2a is a small molecule antagonist/inhibitor.

28. The method of claim 23, wherein the restoring of self-renewal by administration of inhibitor of Cdkn2a is effective to treat or ameliorate a myopathy.

29. The method of claim 23, wherein the restoring of self-renewal by administration of inhibitor of Cdkn2a is effective to treat or ameliorate one or more of a neuromuscular disease or of sarcopenia.

30. The method of claim 23, wherein the restoring of self-renewal by administration of inhibitor of Cdkn2a is effective to treat or ameliorate one or more of spinal muscular atrophy (SMA) and Duchenne Muscular Dystrophy (DMD).

31. The method of claim 23, wherein the inhibitor of Cdkn2a is an oligonucleotide therapeutic agent comprising one or more of SEQ ID NOs: 1-7.

32. A composition for use in preventing premature aging or restoring self-renewal of muscle satellite cells in a subject, the composition comprising an inhibitor of Cdkn2a.

33. The composition of claim 32, wherein the inhibitor of Cdkn2a is one or more oligonucleotide therapeutic agents.

34. The composition of claim 33, wherein the inhibitor of Cdkn2a is one or more of RNAi and antisense oligonucleotides.

35. The composition of claim 34, wherein the inhibitor of Cdkn2a is one or more of siRNAs, shRNAs and Gapmers.

36. The composition of claim 32, wherein the inhibitor of Cdkn2a is a small molecule antagonist/inhibitor.

37. The composition of claim 32, further comprising one or more SMN repletion therapies.

38. The composition of claim 32, wherein the inhibitor of Cdkn2a is an oligonucleotide therapeutic agent comprising one or more of SEQ ID NOs: 1-7.
